(19) United States
(12) Patent Application Publication
(10) Pub. No.: US 2008/0113012 A1
(43) Pub. Date: May 15, 2008

(54) Method for using transdermal system with fentanyl

(75) Inventors: Katalin Tisa-Bostedt, Pullach (DE); Wilfried Fischer, Vagen (DE); Christian Leichs, Miesbach (DE)

Correspondence Address: COHEN, PONTANI, LIEBERMAN & PAVANE, 551 FIFTH AVENUE, SUITE 1210, NEW YORK, NY, 10176, US

(73) Assignee: Hexal AG

(21) Appl. No.: 12/006,879

(22) Filed: Jan. 7, 2008

Related U.S. Application Data
(63) Continuation of application No. 10/379,923, filed on Mar. 5, 2003.

(30) Foreign Application Priority Data
Mar. 6, 2002 (DE) 102 09 724.0

(51) Int. Cl. A61K 9/70; A61K 31/445

(52) U.S. Cl. 424/448; 424/449; 514/329

(57) ABSTRACT

This invention relates to a transdermal system containing fentanyl as the active ingredient and consisting of or comprising a substrate, a mixture of the following ingredients applied to the substrate: the active ingredient,
an oil-based aloe vera extract, a resin, and an adhesive, as well as a layer laminated to the mixture applied to the substrate.

### 1. STATE OF THE ART

Transdermal administration of fentanyl is known for treatment of postoperative pain and pain associated with a tumor (see U.S. Pat. No. 4,588,580). In addition to the general applicability of fentanyl and its derivatives, this patent also describes pharmaceutical forms for transdermal administration. These are reservoir systems, which have a liquid reservoir for the active ingredient with an absorption enhancer on the one hand and also a solid reservoir for the active ingredient on the other hand.

The disadvantages of reservoir systems in general and for fentanyl in particular as a narcotic are as follows:
manufacture is complicated and expensive;
abusive use is easily possible due to reuse of the liquid reservoir contents;
the release controlling element can be destroyed by slight mechanical action;
in addition, it is impossible to make a dosage adjustment by simply varying the area of such a system, because that would destroy the membrane;
there is a high risk of skin irritation due to the use of liquid absorption enhancers;
experience has shown that liquid solvents lead to a reduced adhesive power of the pressure-sensitive adhesive over the storage time.

U.S. Pat. No. 4,806,341 describes microdispersed analgesics such as fentanyl in a non-adhesive polymer disc layer. With this polymer disc layer, the active ingredient solution is dispersed in a hydrophilic solvent such as polypropylene in the form of fine droplets in a hydrophobic polymer. The polymer disc layer is attached to the skin with a pressure-sensitive adhesive, which is applied to the disc layer. Disadvantages include:
manufacture is complicated;
experience has shown that liquid solvents lead to reduced adhesion of the pressure-sensitive adhesive over the storage time;
the system does not follow the contours of the skin and contact is therefore variable, leading to unreliable blood levels;
skin irritation due to the liquid solvent/dispersant.

U.S. Pat. No. 5,091,186 describes biphasic transdermal systems including those for fentanyl, where a period during which the active ingredient is released for 10 to 14 hours is followed by a period during which no active ingredient is released. The disadvantages of this formulation in the case of fentanyl are as follows:
release is not continuous;
this system seems to be more suitable for substances such
   as nitroglycerine, which require a phase during which no active ingredient is released to prevent a tolerance from developing.

U.S. Pat. No. 5,816,939 describes a transdermal system for administration of fentanyl in matrices of pressure-sensitive adhesives containing >5% fentanyl using propylene glycol monolaurate (PGML) as the absorption enhancer. The disadvantage of this system is that approximately 75% of the total dose of fentanyl is released on the first day, so this system is not suitable for use over a period of several days.

U.S. Pat. No. 5,006,342 describes a multilayer laminate for transdermal administration of fentanyl, whereby a layer containing the active ingredient is embedded between two elastic structuring layers. The polymer layer containing the active ingredient may also contain substances such as propylene glycol monolaurate to promote solubility or permeation through the skin. The multilayer laminate is secured in position by a pressure-sensitive adhesive, which also contains absorption enhancers. The disadvantage of this formulation is:
it is complicated to manufacture;
large quantities of absorption enhancers may cause skin irritation;
the system is thick and therefore does not follow the contours of the skin and results in variable contact, which in turn leads to unreliable blood levels.

U.S. Pat. No. 4,911,916 describes a diffusion matrix for use of fentanyl, wherein the diffusion matrix consists of polyurethane foam impregnated with the active ingredient and coated with a pressure-sensitive silicone adhesive.

U.S. Pat. No. 5,719,197 describes a transdermal matrix system which contains active ingredients such as fentanyl in a solution of absorption enhancers, whereby the plasticizing effect of the solvents must be reduced by adding clay minerals. Disadvantages include:
a high potential for causing skin irritation due to the large amounts of absorption enhancers;
processing the composition is difficult due to the addition of clay minerals (sedimentation).

German Patent 196 54 468 describes a transdermal therapeutic system in the form of a patch, which may contain, e.g., fentanyl (column 4 line 21) and aloe vera extract (column 4 line 67) in its adhesive matrix layer which contains the active ingredient. Fentanyl is mentioned in a long list of active ingredients, and the aloe vera extract is mentioned in an equally long list of plant preparations. However, it does not mention a combination of fentanyl and aloe vera extract.

German Patent 197 08 674 describes a transdermal therapeutic system having a backing layer (1) which contains the active ingredient; as the active ingredient it may contain, for example, fentanyl (column 6 line 23), and as the plant preparation it may contain, for example, aloe vera extract (column 6 lines 65/66). Again, fentanyl is included in a practically unlimited list of active ingredients, and aloe vera extract is also listed in a long list of plant preparations. However, this state of the art does not mention a combination of fentanyl and aloe vera extract. It should also be emphasized that this state of the art does not propose to provide a fentanyl system, but instead it provides a transdermal therapeutic system for a practically unlimited number of active ingredients, characterized in that the film layer (3) containing the active ingredient(s) is provided in a sandwich structure between a dividing layer (2) and a protective layer (4), both of which can be removed from the film layer (3) which contains the active ingredient. With regard to the removal of the protective layer (4), see column 4, lines 30/31, and with regard to removal of the dividing layer, see column 4, lines 36/42.

### 2. OBJECT OF THE INVENTION

The object of the present invention is to provide a transdermal system having

| | |
|---|---|
| 1. | a long-lasting (3 to 7 days) and continuous release of active |
| | ingredient of 5 to 200 μg/h per system; |
| 2. | good tolerability by the skin over the entire period of use; |
| 3. | reliable efficacy; |
| 4. | preventing abuse; |
| 5. | good comfort when wearing the patch combined with adequate |
| | adhesive power; |
| 6. | inexpensive production with a highly reproducible quality; |
| 7. | the dosage can be adjusted by simply varying the size of the area; |
| 8. | high stability in storage and |
| 9. | high mechanical stability due to the homogeneous structure of |
| | the system. |

### 3. DESCRIPTION OF THE INVENTION

The object on which this invention is based is achieved by a transdermal system containing fentanyl as the active ingredient, whereby the transdermal system consists of
a substrate,
a mixture of the following ingredients applied to the substrate:
the active ingredient,
an oil-based aloe vera extract,
a resin and
an adhesive, as well as
a layer laminating the applied mixture to the substrate, or the transdermal system may contain the components listed above.

The transdermal system according to this invention may be characterized by a macerate containing soybean oil as the extraction medium, in particular a macerate of leaves of Aloe barbadensis, preferably fresh leaves of Aloe barbadensis as the aloe vera extract.

Furthermore, the transdermal system according to this invention may be characterized by an aloe vera extract containing approximately 7% oil of aloe vera leaves and approximately 93% soybean oil.

Furthermore, the transdermal system according to this invention may be characterized by an ester of colophony, a hydrogenated colophony ester, a synthetic organic resin and/or a synthetic hydrocarbon compound as the resin.

Furthermore, the transdermal system according to this invention may be characterized by a ratio of aloe vera extract to resin in the range of 1:10 to 99:1, preferably 1:5 to 50:1 and in particular 1:2 to 15:1 (based on weight).

Furthermore, the transdermal system according to this invention may be characterized by a thermoplastic elastomer, in particular a pressure-sensitive thermoplastic elastomer as the adhesive, in particular a thermoplastic elastomer based on a block copolymer, preferably a styrene-butadiene-styrene block copolymer (SBS), a styrene-isoprene-styrene block (SIS) or a styrene-ethylene/butadiene-styrene block copolymer (SE/BS) or a hydrocarbon adhesive, preferably an acrylate adhesive or a polyisobutylene adhesive or a silicone adhesive.

Furthermore, the transdermal system according to this invention may be characterized in that it does not have any additional fixative agent except for the components listed above.

Furthermore, the transdermal system according to this invention may be characterized by a fentanyl-to-adhesive ratio in the range of 0.01:1 to 1:1, and preferably 0.1:1.

Furthermore, the transdermal system according to this invention may be characterized by a siliconized plastic film, in particular a siliconized polyester film, a fluorinated plastic film or siliconized paper as the substrate.

Furthermore, the transdermal system according to this invention may be characterized by a plastic film, in particular a polyester film, a nonwoven web, a plastic foam or a fabric as the laminating layer.

Finally, the transdermal system according to this invention may be characterized by a weight per unit of area in the range of 20 to 200 g/m².

### 4. DETAILED DESCRIPTION OF THE INVENTION

In development work, it has been found that a mixture of fentanyl, an oil-based aloe vera extract, a resin and an adhesive achieves all of the goals listed above, regardless of its chemical properties, i.e., namely consisting of thermoplastic elastomers based on block copolymers, e.g., styrene-butadiene-styrene (SBS), styrene-isoprene-styrene (SIS) or styrene-ethylene/butadiene-styrene (SE/BS) or acrylate or polyisobutylene adhesives (hydrocarbon adhesives) or silicone adhesives, in other words, a constant flow lasting at least three days is maintained, while at the same time an optimum adhesive power is observed. This is a transdermal system which is also very thin, namely between 20 and 200 g/m² and can adapt very well to the particular location on the body where it is worn due to its flexibility. Therefore, this ensures that a constant rate of release is maintained. It has surprisingly been found that it is possible to check on the release of the active ingredient by varying the concentration ratio between aloe vera extract and resin. Therefore, it is possible to establish a sufficient flow of fentanyl through the skin to ensure the therapeutic effect. The ratio of aloe extract to resin should amount to a maximum of 0.1 to 99, in particular 0.5 to 15, for example.

Fentanyl base is soluble in a sufficient concentration in the adhesive polymer in a fentanyl-to-adhesive ratio of max. 0.01 to 1:1, e.g., 0.1:1 in this specific case, and the chemical potential of fentanyl in the polymer is high enough, even without adding other components, to maintain a sufficient flow of active ingredient through the intact skin for at least three days.

Comparative Example 1 shows the in-vitro skin permeation of the commercially available reservoir system Durogesic® TTS, in which the permeation of active ingredient through the skin is influenced by ethanol as an absorption enhancer.

Comparative Example 2 shows the in-vitro permeation through the skin from a fentanyl matrix consisting of a styrene-isoprene-styrene adhesive (SIS) without any other additives. The skin permeation is comparable to that obtained in Comparative Example 1. The disadvantage of this system in comparison with Comparative Example 2 is the inadequate adhesive power to ensure a reliable efficacy for three days or seven days.

Due to the addition of the resin, the adhesive power of the transdermal system according to the present invention is so great that an excellent adhesive power is achieved over a three- to seven-day period without any irritation of the skin. No additional fixative agents need be used. Although resins such as Foral E105 (an ester of colophony) lower the rate of release, the oil-based aloe vera extract has the effect of promoting release (see Example 1). Thus, through the choice of a suitable ratio of aloe extract to resin, it is possible to adjust the release of fentanyl from the transdermal systems according to this invention to the level required therapeutically and to maintain an excellent adhesive power in the meantime.

Aloe vera extract is a macerate of fresh leaves of Aloe vera (L.) or Aloe barbadensis (Mill.) with soybean oil as the extraction medium. The extract contains 7% oil of aloe vera leaves and 93% soybean oil. Resins are esters of colophony or hydrogenated colophony esters or synthetic hydrocarbon compounds.

Fentanyl patches can be produced on traditional machine equipment with which those skilled in the art are familiar. Fentanyl base is dissolved or dispersed in a suitable, readily volatile solvent such as methanol, ethanol, isopropanol, dioxane or n-heptane. The solution or dispersion is mixed with a solution of the pressure-sensitive adhesive described above with the addition of aloe vera extract and resin in a suitable vessel, and optionally (but not necessarily) conventional substances such as fillers, skin-protective substances, tackifiers, etc. may be added. The mixture of fentanyl with the adhesive and optionally other substances may be applied to a substrate, e.g., a siliconized or fluorinated plastic film, siliconized paper or the like, usually in the form of a layer in a conventional coating machine, and then freed of solvent in a downstream dryer. In special cases, however, several layers of the same or different composition may also be applied. After leaving the dryer, the active ingredient/adhesive matrix, which is now dried and forms a self-stick layer, may be laminated to another layer, which may be, for example, a plastic film, a nonwoven web, a plastic foam, a fabric or the like.

In another processing step, the desired transdermal systems may be cut or punched out in a defined shaped and size using a cutting or punching device with which those skilled in the art are familiar. The finished systems may be introduced into bags or similar packages to protect them. The matrix of the systems typically contains fentanyl in a concentration range between 0.1% and 20%, preferably between 2% and 10%. The dried matrix usually has a weight per unit of area of between 20 g/m² and 200 g/m², preferably between 50 g/m² and 120 g/m². The rate of release is in the range between 5 and 200 μg fentanyl per hour per system, preferably between 10 μg/h and 100 μg/h per system.

Essentially two methods are used to characterize transdermal systems from the standpoint of their release of the active ingredient:

1. In-vitro skin permeation tests

2. In-vitro release tests according to valid pharmacopoeias.

Skin permeation tests are frequently performed on isolated skin of naked mice. To do so, a piece of TTS patch is attached to the top of the skin and this construction is mounted in a diffusion cell. A buffer solution (acceptor) then comes in contact with the underside of the skin, and the change in concentration in the acceptor medium is measured as a function of time, e.g., by using a high-pressure liquid chromatographic method of analysis. The results obtained with the preparations according to this invention are listed in the following examples.

The in-vitro release tests are performed in glass containers constructed in accordance with the specifications of the pharmacopoeias. The patch is attached to a screen plate in a cylindrical one-liter container having a round bottom so that the adhesive layer is facing upward. The screen plate is placed on the bottom of the container, the container is filled with water and stirred with a defined stirrer to equalize the concentration. The time-dependent concentration in the release medium is again measured here. The results of these tests are given in the examples. The difference between these methods is that the release tests describe the release behavior of the active ingredient from the patch, but this does not usually correlate with the biological effect. However, the skin permeation model also includes the step of distribution of the active ingredient into the skin and diffusion through the skin in addition to the required release from the patch. As a rule, this permits correlations with the biological effect.

### 5. EXAMPLES

### Comparative Example 1

Durogesic® TTS, a commercial fentanyl patch has the following characteristics (according to U.S. Pat. No. 4,588,580):

| | | |
|---|---|---|
| fentanyl content: | 2.5 | mg |
| area: | 10 | cm² |

Composition (Qualitative):
silicone adhesive
ethanol/water
hydroxyethylcellulose
microporous ethylene-vinyl acetate membrane (EVA)

This patch was subjected to an in-vitro skin permeation test in a mouse skin model.

Table 1 shows the results.

Skin Permeation

A piece of skin measuring 1.5 cm² from a female naked mouse was freed of subcutaneous tissue and placed on the opening, which measured exactly 1 cm², of an automated diffusion cell, attached with adhesive to a piece of fentanyl patch approximately 1.5 cm in size and sealed on the cell with a pressing device. This cell was filled with 15 mL of a physiological HEPES buffer solution and regulated at a temperature of 32° C. After defined periods of time, samples of the buffer solution were taken and the active ingredient content in the samples was determined by means of high-pressure liquid chromatographic (HPLC) analytical method. All the patches described below were tested by this method. The results are shown in Table 1.

### Comparative Example 2

A matrix fentanyl patch was prepared as the comparison using an SIS adhesive without adding resin or aloe vera oil. The system had the following characteristics:

| | | |
|---|---|---|
| fentanyl content: | 4.5 | mg |
| area: | 10 | cm² |

Composition:

| | | |
|---|---|---|
| SIS (styrene-isoprene-styrene) | 85.5 | mg |
| Duro-Tak* 387-6173: | | |
| siliconized polyester film FL200075 1S**: | 10 | cm² |
| polyester film Hostaphan MN 19 MED***: | 10 | cm² |

*National Starch & Chemical, Zutphen, Netherlands
**Loparex, Apeldoorn, Netherlands
***Mitsubishi Polyester Foils, Frankfurt, Germany

Preparation

Fentanyl is dissolved in ethanol. The solution is added to a sufficient amount of the commercial adhesive solution and homogenized using an agitator. Using a drawing doctor, the homogeneous solution is applied as a coating to a sheet of siliconized polyester film (approximately 75 μm) in a defined layer thickness. The sheet is then dried in a drying cabinet for 30 minutes at 50° C. until dry. Then an approximately 19 μm-thick polyester film is laminated onto the adhesive layer. Using a hand punch, 10 cm² patches are then punched out of the finished laminate.

Skin Permeation

See Comparative Example 1. The results are shown in Table 1.

### Comparative Example 3

A fentanyl patch was produced using an SIS adhesive to which aloe vera oil was added as a comparison. The system had the following characteristics:

| | | |
|---|---|---|
| fentanyl content: | 4.5 | mg |
| area: | 10 | cm² |

Composition:

| | | |
|---|---|---|
| SIS (styrene-isoprene-styrene) | 81 | mg |
| Duro-Tak* 387-6173: | | |
| aloe vera oil: | 4.5 | mg |
| siliconized polyester film FL200075 1S**: | 10 | cm² |
| polyester film Hostaphan MN 19 MED***: | 10 | cm² |

*National Starch & Chemical, Zutphen, Netherlands
**Loparex, Apeldoorn, Netherlands
***Mitsubishi Polyester Foils, Frankfurt, Germany

Preparation

See Comparative Example 2.

Skin Permeation

See Comparative Example 1. The results are summarized in Table 1.

### Comparative Example 4

A fentanyl patch was prepared using an SIS adhesive to which resins were added. The system had the following characteristics:

| | | |
|---|---|---|
| fentanyl content: | 4.5 | mg |
| area: | 10 | cm² |

Composition:

| | | |
|---|---|---|
| SIS (styrene-isoprene-styrene) | 76.5 | mg |
| Duro-Tak* 387-6173: | | |
| resin (Foral 105E): | 9 | mg |
| siliconized polyester film FL200075 1S**: | 10 | cm² |
| polyester film Hostaphan MN 19 MED***: | 10 | cm² |

Preparation

See Comparative Example 2.

Skin Permeation

See Comparative Example 1. The results are summarized in Table 1.

TABLE 1
In-vitro skin permeation of fentanyl matrix patches

| | Comp. | Comp. | Comp. | Comp. | |
|---|---|---|---|---|---|
| Time | Ex. 1 | Ex. 2 | Ex. 3 | Ex. 4 | Example |
| (hours) | fentanyl permeation (□g/cm²) | | | | |
| 6 | 26.18 | 24.2 | 22.4 | 14.6 | 35.9 |
| 15 | 62 | 67.2 | 83.7 | 46.9 | 88.4 |
| 24 | 99.8 | 109.3 | 147.9 | 81.1 | 133.8 |
| 36 | 147.4 | 160 | 227.1 | 123.2 | 178.9 |
| 48 | 190.4 | 204.6 | 298.6 | 160 | 214.7 |
| 60 | 226.8 | 240.3 | 353.2 | 188.6 | 240.3 |

### Example 1

A fentanyl patch according to this invention using a combination of aloe vera extract and a resin has the following characteristics:

| | | |
|---|---|---|
| fentanyl content: | 5 | mg |
| area: | 10 | cm² |

Composition:

| | | |
|---|---|---|
| polyisobutylene PSA (MA24A): | 61.7 | mg |
| aloe vera extract | 7.5 | mg |
| resin (Foral 105E) | 0.75 | mg |
| siliconized polyester film FL200075 1S**: | 10 | cm² |
| polyester film Hostaphan MN 19 MED***: | 10 | cm² |

Preparation

See Comparative Example 2.

Skin Permeation

See Comparative Example 1. The results are summarized in Table 1.

### 6. DISCUSSION

Comparative Example 1 in Table 1 shows the results of a skin permeation test after 60 hours; these results were obtained using the comparative preparation Durogesic® TTS with 227 μg fentanyl/cm². Comparative Example 2 shows the results of a simple matrix system using the adhesive described here without any additional additives with 240 μg/cm² after 60 hours. It also shows as the basic recipe a comparable but somewhat greater permeation after three days than that in Comparative Example 1. However, this formulation is not suitable for several days of use because the adhesive power is not sufficient. Therefore, Comparative Example 4 shows the skin permeation of a formulation having very good adhesive properties obtained by adding colophony resin. However, the permeation of fentanyl here drops below that of Comparative Example 1. Comparative Example 3 shows a formulation which contains an oil-based aloe vera extract as the substance that promotes release and thus increases the amount of fentanyl permeating through the skin by approximately 50% in comparison with Comparative Example 2. To achieve optimum fentanyl permeation for therapeutic use, a resin from Comparative Example 4 (Foral E 105) is added to this formulation so that adequate adhesive power for three to seven days is ensured (see Example 1); in this case, the quantities permeating through the skin drop to approximately the levels obtained in Comparative Example 1, but this is still sufficient for the desired therapeutic effect.

1-15. (canceled)

16. A method for treating pain in a subject comprising affixing to the subject's skin a transdermal system comprising a substrate, a mixture applied to the substrate and a layer laminated onto the applied mixture, wherein the applied mixture comprises fentanyl as an active ingredient, an oil-based aloe vera extract, a resin, and an adhesive.

17. The method of claim 16 wherein the oil-based aloe vera extract is obtained by using soy oil as an extraction agent.

18. The method of claim 16 wherein the oil-based aloe vera extract comprises a macerate of leaves of Aloe barbadensis.

19. The method of claim 16 wherein the oil-based aloe vera extract comprises a macerate of fresh leaves of Aloe barbadensis.

20. The method of claim 16 wherein fentanyl has a concentration of 0.1 to 20 weight percent in the applied mixture.

21. The method of claim 16 wherein fentanyl has a concentration of 2 to 10 weight percent in the applied mixture.

22. The method of claim 16 wherein the aloe vera extract contains about 7 weight percent oil of aloe leaves and about 93 weight percent of soy oil.

23. The method of claim 16 wherein the resin is selected from the group consisting of an ester of colophony, a hydrogenated colophony ester, a synthetic organic resin and a synthetic hydrocarbon compound.

24. The method of claim 16 wherein the weight ratio of aloe vera extract to the resin is in the range of 0.1:1 to 99:1.

25. The method of claim 16 wherein the weight ratio of aloe vera extract to the resin is in the range of 0.2:1 to 50:1.

26. The method of claim 16 wherein the weight ratio of aloe vera extract to the resin is in the range of 0.5:1 to 15:1.

27. The method of claim 16 wherein the adhesive is a thermoplastic elastomer.

28. The method of claim 16 wherein the adhesive is a pressure-sensitive thermoplastic elastomer.

29. The method of claim 16 wherein the adhesive is selected from the group consisting of a thermoplastic elastomer based on a block copolymer, a hydrocarbon adhesive, and a silicone adhesive.

30. The method of claim 29 wherein the block copolymer is selected from a group consisting of a styrene-butadiene-styrene block copolymer, a styrene-isoprene-styrene block copolymer and a styrene-ethylene/butadiene-styrene block copolymer.

31. The method of claim 29 wherein the hydrocarbon adhesive is selected from the group consisting of an acrylate adhesive or a polyisobutylene adhesive.

32. The method of claim 16 wherein the transdermal system does not contain any additional fixative.

33. The method of claim 16 wherein the weight ratio of fentanyl to the adhesive is in the range of 0.1:1 to 1:1.

34. The method of claim 16 wherein the weight ratio of fentanyl to the adhesive is 0.1:1.

35. The method of claim 16 wherein the substrate is a removable protective layer selected from the group consisting of a siliconized plastic film, a fluorinated plastic film and siliconized paper.

36. The method of claim 35 wherein the siliconized plastic film is a siliconized polyester film.

37. The method of claim 16 wherein the laminating layer is selected from the group consisting of a plastic film, a nonwoven web, a plastic foam and a fabric.

38. The method of claim 37 wherein the plastic film is a polyester film.

39. The method of claim 16 wherein the laminating film is a covering layer, which is impermeable with respect to the active ingredient.

40. The method of claim 16 wherein the transdermal system has a density of 20 to 200 g/m².

41. The method of claim 16 wherein the transdermal system has a density of 50 to 120 g/m².

42. The method of claim 16, wherein the transdermal system further comprises a filler.

43. The method of claim 16 wherein the transdermal system further comprises a skin-protective agent.

44. The method of claim 16 wherein the transdermal system further comprises a tackifying agent.

45. The method of claim 16, wherein the transdermal system consists of a substrate, a mixture applied to the substrate and a layer laminated onto the applied mixture, wherein the applied mixture consists of fentanyl as an active ingredient, an oil-based aloe vera extract, a resin, and an adhesive.

46. The method of claim 16, wherein the transdermal system causes the administration of fentanyl to the subject over a period of from three days to seven days and in an amount of from 5 μg/hr to 200 μg/hr.
